(19) United States
(12) Patent Application Publication
(10) Pub. No.: US 2025/0163524 A1
(43) Pub. Date: May 22, 2025

(54) RAPID TARGET GENE DETECTION METHOD USING PLASMONIC PHOTOTHERMAL REACTION

(71) Applicant: Soonchunhyang University Industry Academy Cooperation Foundation, Asan-si (KR)

(72) Inventor: Jung-Hoon LEE, Seoul (KR)

(73) Assignee: Soonchunhyang University Industry Academy Cooperation Foundation, Asan-si (KR)

(21) Appl. No.: 18/583,776

(22) Filed: Feb. 21, 2024

(30) Foreign Application Priority Data
Nov. 17, 2023 (KR) 10-2023-0159846

(51) Int. Cl. C12Q 1/70; C12Q 1/6851

(52) U.S. Cl. CPC C12Q 1/701; C12Q 1/6851

(57) ABSTRACT

The present invention relates to a plasmon-based target gene detection method. The present invention involves performing an RT-PCR reaction using plasmonic nanoparticles, adding SYBR Green I and 3,3′,5,5′-tetramethylbenzidine, and visually detecting the presence of a target gene by irradiating an LED. The present invention solves the problem of the related art in which it was not easy to visually detect a target gene, thereby detecting the target gene with excellent sensitivity and specificity in a short period of time.

### STATEMENT REGARDING PRIOR DISCLOSURES BY THE INVENTOR OR A JOINT INVENTOR

Applicant designates the following article as a grace period publication in order to expedite examination of the application in accordance with 37 CFR 1.77 (b) (6) and MPEP 608.01 (a): “Next-Generation Diagnostic Test for Dengue Virus Detection Using an Ultrafast Plasmonic Colorimetric RT-PCR Strategy” published in Analytica Chimica Acta, volume 1274, on Jun. 26, 2023. The disclosures of the article are incorporated herein by reference in their entirety for all purposes.

### CROSS-REFERENCE TO RELATED APPLICATION

This application claims priority to and the benefit of Korean Patent Application No. 10-2023-0159846, filed on Nov. 17, 2023, the disclosure of which is incorporated herein by reference in its entirety.

### BACKGROUND

### 1. Field of the Invention

The present invention relates to a real-time polymerase chain reaction of a target gene using plasmonic nanoparticles. By applying plasmonic nanoparticles to a real-time polymerase chain reaction, the present invention may not only detect the presence of a target gene through a colorimetric reaction, but also improve the detection specificity and analytical specificity of the target gene. When the polymerase chain reaction according to the present invention is used, simple and rapid detection of the target gene is possible, thereby providing a diagnostic analysis method that can be distributed in the field.

The present invention was derived from an individual basic research project of the Ministry of Science and Information & Communications Technology (ICT) [Project Serial Number: 1711186785, Project Number: NRF-2022R1F1A1070162, Research Project Name: Development of on-site ultra-fast real-time PCR platform based on multifunctional plasmonic photothermal magnetic nanoparticles], Science and Engineering Academic Research Base Construction Project of the Ministry of Education [Project Serial Number: 1345362911, Project Number: NRF-2021R1A6A1A03039503, Research Project Name: Korea Native Animal Resource Utilization Convergence Research Institute], ‘Regional Innovation Mega Project’ Project of the Ministry of Science and ICT [Project Serial Number: 20230007, Project Number: 2023-DD-UP-0007, Research Project Name: Development of source technology for meta-platformization of marine bio strategic materials].

### 2. Discussion of Related Art

The polymerase chain reaction (PCR) method is a test method used in almost all processes of performing an experiment by manipulating a genetic material, that is, a method of amplifying a specific target genetic material desired to be detected. Because the polymerase chain reaction can amplify a large amount of genetic material having the same nucleotide sequence from a small amount of genetic material, it is used to amplify human DNA to diagnose various types of genetic diseases. Also, the polymerase chain reaction is used in the diagnosis of infectious diseases and the like because it is applied to the DNA of bacteria, viruses, and fungi.

Meanwhile, dengue virus is a single-stranded positive-sense RNA virus that belongs to the genus Flavivirus of the family Flaviviridae. Dengue fever and dengue hemorrhagic fever are acute febrile diseases that develop when an Aedes aegypti or Aedes albopictus mosquito infected with the dengue virus bites a person.

Over the past few decades, dengue fever infections have spread rapidly within tropical and subtropical countries, putting nearly one third of the world's population at risk. Today, it is one of the most important public health issues in many countries with limited resources, threatens human life in many ways, and puts enormous pressure on health care systems.

More importantly, the combined impact of the COVID-19 pandemic and the dengue fever epidemic could have potentially devastating consequences for patients in highly endemic areas, who have multiple serotypes of DENV. Effective vaccines and early-stage diagnosis of dengue virus (DENV) infection are important to improve clinical outcomes and prevent the spread of the disease. Simple, cost-effective, and sensitive diagnostic tests for use in the field are essential to control the disease, especially in remote areas with limited access to health care providers and facilities. However, a diagnostic tool having these functions has not yet been developed.

Accordingly, the present inventors have developed a test method to detect genes in real time using plasmonic nanoparticles. Unlike existing methods, it was confirmed that a target gene may be detected by a color change, and the detection is possible with high sensitivity and specificity in a short period of time. Therefore, the present invention has been completed based on the finding.

### SUMMARY OF THE INVENTION

The present invention is directed to providing a method of performing a plasmonic photothermal (PPT)-reverse transcription-colorimetric polymerase chain reaction (RTcPCR) for rapid molecular diagnosis of viral infection.

The present invention is also directed to providing a composition for plasmon-based reverse transcription polymerase chain reaction analysis, and a kit for detecting a target gene using the same.

Hereinafter, the present invention will be described in detail. Advantages and features of the present invention and methods of achieving the same will become clear with reference to embodiments described below. However, the present invention is not limited to the embodiments disclosed below, and may be implemented in various different forms. The present embodiments are merely intended to make the disclosure of the present invention complete and fully inform the scope of the present invention to those skilled in the art to which the present invention pertains. Thus, the present invention is only defined by the scope of the claims. Throughout this specification, like reference numerals refer to like elements.

Unless otherwise defined, all terms (including technical and scientific terms) used in this specification may be used with meanings that can be commonly understood by those skilled in the art to which the present invention pertains. Also, the terms defined in commonly used dictionaries are not ideally or excessively interpreted unless clearly specifically defined. The terminology used in this specification is for describing the embodiments and is not intended to limit the present invention. In this specification, singular forms also include plural forms, unless specifically stated otherwise in the context.

According to an aspect of the present invention, there is provided a method of performing a plasmon-based reverse transcription polymerase chain reaction for detecting a target gene, which includes i) mixing a target gene, a primer set, and plasmonic nanoparticles, and then performing a reverse transcription polymerase chain reaction; and ii) mixing 3,3′,5,5′-tetramethylbenzidine with the product obtained in step i). SYBR Green I may be added in step i) or ii) of the plasmon-based reverse transcription polymerase chain reaction according to the present invention. That is, SYBR Green I may be added before or after the polymerase chain reaction.

In the polymerase chain reaction of the present invention, plasmonic nanoparticles, 3,3′,5,5′-tetramethylbenzidine (hereinafter also referred to as “TMB”), and SYBR Green I (hereinafter also referred to as “SGI”) may be added to induce a photothermal reaction of nanoparticles. To induce a photothermal reaction, the polymerase chain reaction of the present invention may further include iii) irradiating a blue LED after step ii). When the target gene is present in the reaction product, the reaction product develops a blue color when the reaction product is irradiated with the blue LED. Therefore, when the product according to step iii) develops a blue color, it may be determined that the target gene is present.

In the present invention, the plasmonic nanoparticles may be metal particles, and preferably may have a core-shell structure consisting of a core including iron oxide (FeO); and a shell including gold (Au) attached to a surface of the core. Polyethylene glycol may be bound to the gold particles of the present invention.

The target gene to be detected according to the polymerase chain reaction of the present invention may include all genes that may be amplified by the polymerase chain reaction, and is preferably a viral gene.

The viral gene of the present invention includes viral genes derived from dengue virus, picornavirus, flavivirus, Zika virus, Powassan virus, Chikungunya virus, enterovirus, respiratory syncytial virus (RSV), Rift Valley fever, influenza virus, Tacaribe virus, Mayaro virus, West Nile virus, Yellow fever virus, and coronavirus, but the present invention is not limited thereto.

According to another aspect of the present invention, there is provided a composition for plasmon-based reverse transcription polymerase chain reaction analysis, which includes plasmonic nanoparticles, SYBR Green I, and 3,3′,5,5′-tetramethylbenzidine.

In the present invention, the SYBR Green I is preferably added before or after the polymerase chain reaction, and the 3,3′,5,5′-tetramethylbenzidine is preferably added after the polymerase chain reaction.

According to still another aspect of the present invention, there is provided a kit for detecting a target gene, which includes the composition for plasmon-based reverse transcription polymerase chain reaction analysis. The detection kit of the present invention develops a blue color when the target gene is present, which makes it possible to detect the target gene with the naked eye.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic diagram showing the overall flow of a method of detecting a target gene using a plasmonic photothermal reaction-based RTcPCR (PPT-RTcPCR) platform according to the present invention;

FIGS. 2A-2B show the workflow of the plasmonic photothermal reaction-based RTcPCR (PPT-RTcPCR) platform. FIG. 2A is a flowchart of the PPT-RTcPCR platform in which PPT-based RT-PCR is combined with colorimetric detection, and FIG. 2B shows a colorimetric reading method using a plasmonic photothermal reaction-based RTcPCR (PPT-RTcPCR). Referring to FIG. 2B, amplification does not occur in a negative sample in which a target gene is not present, and little color change occurs because TMB cannot be oxidized when no dsDNA-SGI complex is formed. On the other hand, in a positive sample in which the target gene is present, amplicons increase exponentially, in which case TMB may be further oxidized by the dsDNA-SGI complex, thereby causing a noticeable color change from colorless to blue;

FIGS. 3A-3I show the results showing the characterization and photothermal performance of plasmonic nanoparticles (PMNs) prepared according to the present invention. FIG. 3A is a TEM image of plasmonic nanoparticles (PMNs) having a core-shell structure. FIG. 3B shows a high-angle annular dark-field (HAADF) image and an elemental mapping image of the plasmonic nanoparticles (PMNs), and FIG. 3C shows the ultraviolet-visible (UV-Vis) spectrum values of the plasmonic nanoparticles (PMNs). FIG. 3D shows the magnetization curve of the plasmonic nanoparticles (PMNs), and FIG. 3E shows the temperature profile of a solution according to the presence of plasmonic nanoparticles (PMNs) during lighting treatment. FIG. 3F is a graph confirming the effect of a concentration of the plasmonic nanoparticles (PMNs) on heating and cooling ramp speeds. The optimal ramp speed was observed for 16 OD plasmonic nanoparticles (PMNs). FIG. 3G shows a representative temperature profile of PPT-based RT-PCR. Thermocycling started at 50° C. (5 minutes), followed by repeating 40 cycles at 90° C. (0 second) and 60° C. (8 seconds). FIG. 3H shows an enlarged temperature profile for isothermal reverse transcription, and FIG. 3I shows an enlarged temperature profile for PCR amplification;

FIGS. 4A-4C show the results of evaluating the analytical specificity and specificity of the PPT-RTcPCR platform according to the present invention. FIGS. 4A and 4B show the results of comparing quantitative curves between PPT-RTcPCR and RT-qPCR using RNA extracted from IVT DENV RNA and DENV2-spiked serum samples, respectively. FIG. 4C shows the results of analyzing the absorbance for DENV2 and other non-specific targets. In FIG. 4, the RT-qPCR results are expressed as Ct values, which are inversely proportional to the logarithm of the copy number/μL of DENV2 RNA; and

FIGS. 5A-5C show the results of clinically verifying the PPT-RTcPCR platform. FIG. 5A shows a heat map of PPT-RTcPCR results for 131 positive samples and 27 negative samples. FIG. 5B is an image analyzing the absorbance of the clinical test results. In FIG. 5B, the samples were classified using a predefined threshold of 0.53 and an optimal ROC threshold of 0.51. FIG. 5C shows the results of analyzing the ROC curve of clinical outcomes. In FIG. 5C, the AUC is 0.99.

### DETAILED DESCRIPTION OF EXEMPLARY EMBODIMENTS

Hereinafter, various embodiments are presented to aid in understanding the present invention. However, it should be understood that the following examples are merely provided to make the invention easier to understand, and not intended to limit the protection scope of the invention.

The present invention provides a method for performing a polymerase chain reaction using plasmonic nanoparticles.

The polymerase chain reaction of the present invention is a (reverse transcription, colorimetric) polymerase chain reaction based on a plasmonic photothermal reaction, and is hereinafter referred to as PPT-RTcPCR.

The PPT-RTcPCR reaction of the present invention may include i) mixing a target gene, a primer set, and plasmonic nanoparticles, and then performing a reverse transcription polymerase chain reaction; and ii) mixing 3,3′,5,5′-tetramethylbenzidine with the product obtained in step i). In this case, SYBR Green I may be added in step i) or ii).

In the present invention, the term “PPT” has the same meaning as the term “plasmonic photothermal.” In the present invention, the term “PPT-” refers to various reactions based on the photothermal reaction of plasmonic nanoparticles.

In the present invention, the term “plasmonic nanoparticles (PMNs)” refers to nano-sized particles that exhibit plasmon resonance.

Metals that exhibit a plasmonic resonance effect may be used as the plasmonic nanoparticles of the present invention. As a specific example, gold (Au) nanoparticles, silver (Ag) nanoparticles, or a combination of gold (Au) and silver (Ag) nanoparticles may be used.

According to one preferred embodiment of the present invention, the plasmonic nanoparticles have a core-shell structure consisting of a core including iron oxide (FeO); and a shell including gold (Au) attached to a surface of the core. Because polyethylene glycol may be bound to the gold particles of the plasmonic nanoparticles, the plasmonic nanoparticles may be configured so that the polyethylene glycol can be expressed on the outside of the core-shell structure.

In the present invention, the term “target gene” refers to a nucleic acid sequence to be detected, and is annealed or hybridized with a probe under hybridization conditions. The term “target gene” is used interchangeably with the term “target nucleic acid.” In the present invention, the target nucleic acid may be a gene derived from animals, plants, bacteria, viruses, fungi, and the like, or a mutated gene accompanying a genetic disease. In the present invention, the target gene refers to a fragment of a nucleic acid such as DNA, RNA, or the like. In this case, the target gene may be single- or double-stranded DNA or RNA, and may be DNA or RNA that may represent a sense or antisense strand. Also, dsDNA, ssDNA, mixed ssDNA, mixed dsDNA, ssDNa, dsDNA, mRNA, RNA, tRNA, snRNA, or miRNA may be used, but the target gene of the present invention is most preferably in the form of RNA.

In the present invention, the “target gene” is preferably a viral gene. The types of viral genes to be detected in the present invention are not limited, but include viral genes derived from dengue virus type 1-4 (DENV1-4), picornavirus (CVB3), flavivirus, Zika virus, Powassan virus, Chikungunya virus, enterovirus, respiratory syncytial virus (RSV), Rift Valley fever, influenza virus, Tacaribe virus, Mayaro virus, West Nile virus, Yellow fever virus, and coronavirus.

In the present invention, the term “detection” means that a target gene is detected through photothermal reaction-based PCR of the plasmonic nanoparticles of the present invention.

The product of the PPT-RTcPCR reaction according to the present invention may be irradiated with a blue LED. When the target gene is present in the product, a blue color appears in the reaction product when the reaction product is irradiated with a blue LED. Through this colorimetric reaction, when a blue color appears in the reaction product, it is confirmed that the target gene is present, which is judged to be “positive.”

In the present invention, the term “complementary binding site” refers to a site that may form a complementary base pair between nucleotide sequences.

In the present invention, the term “primer” refers to a nucleic acid sequence with a short free 3′ hydroxyl group, that is, a short nucleic acid sequence that may form a base pair with a complementary nucleic acid template and serves as a starting point for copying strands from the nucleic acid template. Primers may initiate DNA synthesis in the presence of four different nucleoside triphosphates and reagents for polymerization (i.e., DNA polymerase or reverse transcriptase) in an appropriate buffer at an appropriate temperature.

When designing the primers, there are several restrictions, such as the A, G, C, and T content ratio of the primers, prevention of primer-dimer formation, and prohibition of repeating the same base sequence three times or more. In addition, in the single PCR reaction, conditions such as the amount of template DNA, the concentration of primers, the concentration of dNTP, the concentration of Mg²⁺, the reaction temperature, and the reaction time, and the like should be appropriate.

The primers may incorporate additional characteristics without changing the basic properties. That is, the nucleic acid sequence may be modified using many means known in the art. Examples of such modifications may include methylation, capping, substitution of a nucleotide with one or more homologs, and modification of nucleotides into uncharged linkers such as phosphonates, phosphotriesters, phosphoroamidates, carbamates, or the like, or charged linkers such as phosphorothioates, phosphorodithioates, or the like. Also, the nucleic acid may have one or more additional covalently linked residues such as nucleases, toxins, antibodies, signal peptides, proteins (such as poly L lysine), intercalating agents (such as acridine, psoralen, or the like), chelating agents (such as metals, radioactive metals, and iron oxidizing metals, and the like), alkylating agents, and the like.

Hereinafter, examples of the present invention are provided to aid in understanding the invention. However, it should be understood that the following examples are not intended to limit the protection scope of the invention.

### Example 1

### Materials and Experimental Methods

### 1-1. Preparation of In Vitro Transcribed RNA

The analytical performance of a dengue virus (DENV)-specific PPT-RTcPCR assay was first evaluated using in vitro transcribed (IVT) RNA. A partial sequence of the E gene of a DENV-2 NGC strain (nt 1,453 to 1,550; GenBank Accession AF038403) was synthesized, and cloned into a pGEM-3Z vector downstream of a T7 promoter sequence. The resulting plasmid pGEM-3Z-DENV was linearized by SalI restriction digestion, and then used as a template for in vitro transcription using an mMESSAGE mMACHINE™ T7 transcription kit (Invitrogen™, USA). After residual RNA was removed using TURBO DNase (Invitrogen™, USA), an aliquot of disposable IVT RNA was stored at −80° C. Thereafter, the analytical sensitivity of PPT-RTcPCR was determined using 10-fold serial dilutions of IVT RNA (1 to 10⁶ copies/μL). The concentration of IVT RNA was calculated using a known method.

### 1-2. Nucleic Acid Extraction

For an analytical sensitivity assay, viral RNA was extracted from TCF containing 1.15×10⁶ focus forming units (FFU)/mL of DENV2, and subjected to 10-fold serial dilutions (10¹ to 10⁸) in nuclease-free water.

For an analytical specificity assay, viral RNA was also extracted from a TCF sample containing DENV1, DENV4, ZIKV, CVB3, or FIPV. An archived human plasma sample was used for nucleic acid extraction without further dilution.

All the samples were extracted using a MagMAX™-96 Viral RNA isolation kit (Applied Biosystems, USA) on the KingFisher Flex system (Thermo Fisher, USA). After the extraction step, nucleic acids were eluted with 90 μL of nuclease-free water, and all the nucleic acids were stored at −80° C. for further use.

### 1-3. DENV-Specific PPT-RTcPCR and Optimization

DENV-specific PPT-RTcPCR was developed with some modifications from previously known information. Target DENV2 RNA was first amplified via PPT-based RT-PCR in the presence of plasmonic magnetic nanoparticles (PMNs).

The reaction mixture included 5 μL of a 2× reaction mix, 0.2 μL of a SuperScript™ III RT/Platinum™ Taq mix, 0.1 μL of 50 μM forward (5′-CAG GCT ATG GCA CYG TCA CGA T-3′) and reverse (5′-CCA TYT GCA GCA RCA CCA TCT C-3′) primers, 2 μL of plasmonic nanoparticles (PMNs) having an optical density (OD) of 80, 1 μL of a viral nucleic acid, 1.6 μL of nuclease-free water, and 20 μL of mineral oil. The thermocycling protocol started at 50° C. for 5 minutes, followed by repeating 40 cycles between 90° C. (0 second) and 60° C. (8 seconds). After the PPT-based RT-PCR, 10 μL of a colorimetric solution containing a 2-(N-morpholino) ethanesulfonic acid (MES) buffer (8 μL, 0.1 M), 40 mM TMB (3,3′,5,5′-tetramethylbenzidine, 1 μL), and 80×SGI (SYBR Green I, 1 μL) was added to the reaction solution.

By making use of the magnetic properties of PMNs, the particles were collected with a magnet before being irradiated with a blue light LED (300 mA, 14 V) for 2 minutes. With the photocatalytic activity of the dsDNA-SGI complex, TMB was oxidized under the excitation of a blue light LED by adjusting the pH value of the solution to approximately 5. The absorbance of the resulting mixture was measured using a NanoDrop™ OneC Microvolume UV-Vis Spectrophotometer (Thermo Scientific™).

Reference real-time DENV2 RT-qPCR was performed using a modified method known in the art. Briefly, the reaction mixture included 5 μL of a 2× reaction mix, 0.2 μL of a SuperScript™ III RT/Platinum™ Taq mix, 0.1 μL of 50 μM forward and reverse primers, 0.18 μL of a 10 μM probe (5′-FAM-CTC YCC RAG AAC GGG CCT CGA CTT CAA-BHQ1-3′), 1 μL of a viral nucleic acid, and 3.42 μL of nuclease-free water.

RT-qPCR was performed using a CFX Opus 96 Real-Time PCR system (Bio-Rad) with the following protocol: 50° C. for 30 minutes, 95° C. for 2 minutes, and then 40 cycles at 95° C. for 15 seconds and 60° C. for 1 minute. In the case of the reference real-time RT-PCR, the samples having a Ct value greater than 37 were considered negative because it was difficult to confirm the amplification outcomes.

### Example 2

### Outcomes

### 2-1. PPTRTcPCR Platform Procedure

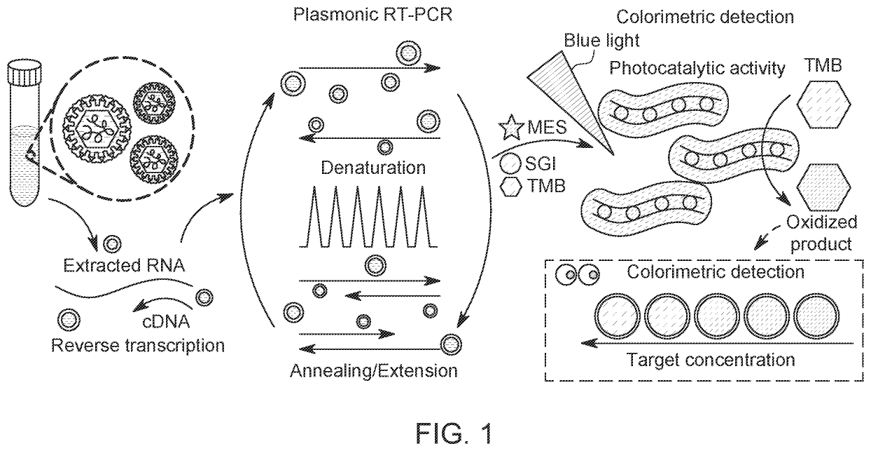

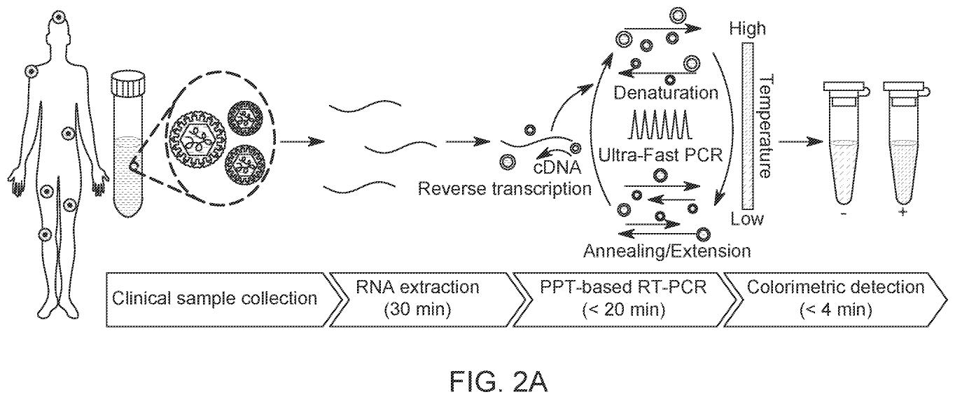

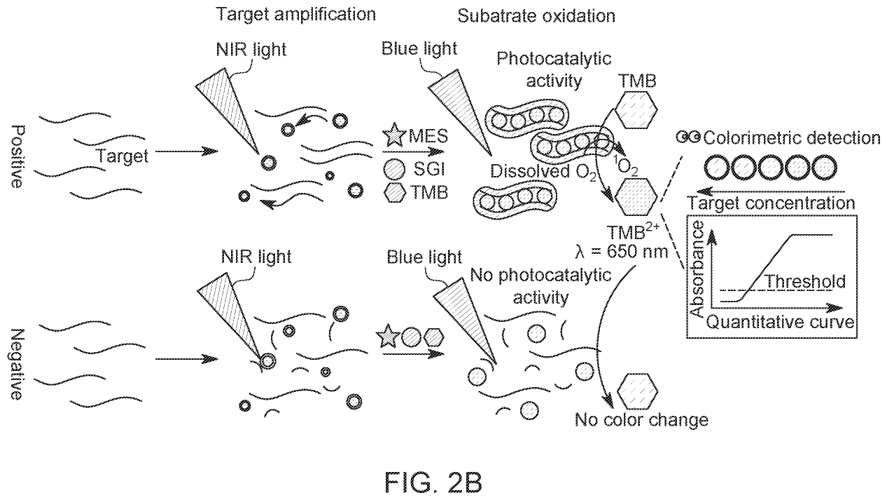

In the present invention, the entire procedure of DENV nucleic acid detection using the PPTRTcPCR platform, from sample collection to target detection, is shown in FIG. 1.

First, the viral RNA extracted from plasma samples of patients suspected of dengue fever infection was subjected to a DENV-specific PPT-RTcPCR reaction. In this system, PMNs were employed as a nano-sized heater for uniform heating. When an infrared (IR)-LED is turned on, the reaction temperature may be easily and precisely controlled by adjusting the light intensity. The heating protocol begins with an isothermal process for reverse transcription of RNA into complementary DNA (cDNA), followed by two-step thermocycling for cDNA denaturation and specific target extension.

Next, amplicons are visually detected using a TMB oxidation-based colorimetric strategy by adding the colorimetric solution containing an MES buffer, SGI, and TMB. The amplified dsDNA is a reactant for forming a photocatalyst, and is triggered only when the SGI is intercalated in the dsDNA. Upon the irradiation with a blue light LED, singlet oxygen is generated by energy transfer from SGI to dissolved oxygen in the solution, which subsequently induces TMB oxidation. The reaction product changes from colorless to a blue color, thereby making the generation of amplicons visible. Without viral RNA, there were no specific RT-PCR amplification, no formation of the dsDNA-SGI complex, and thereby no oxidation of TMB, so that the solution remains colorless.

Such a simple design enables this device to be developed into a POC diagnostic system.

### 2-2. Characterization of PPT-RTcPCR Platform

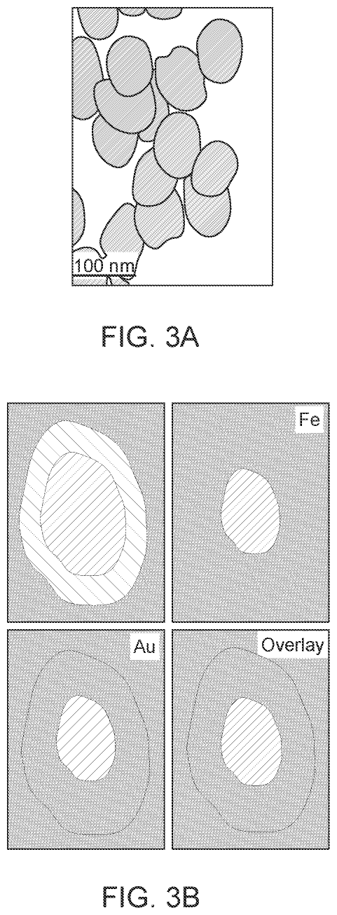

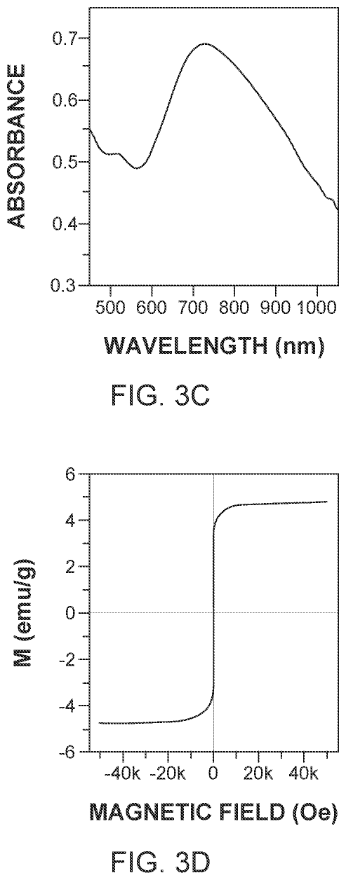

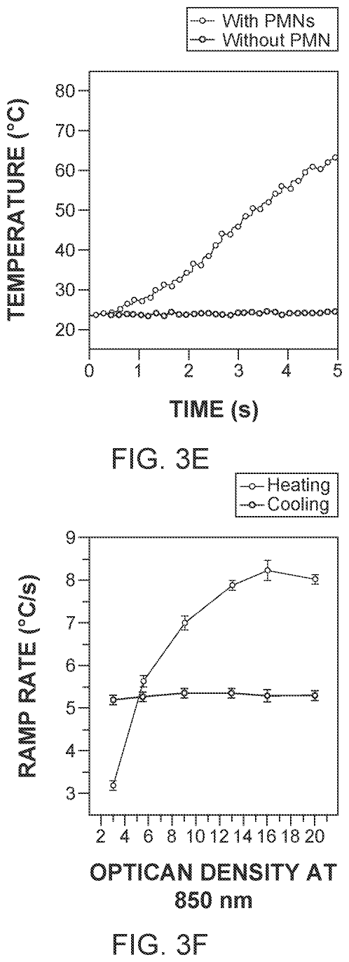

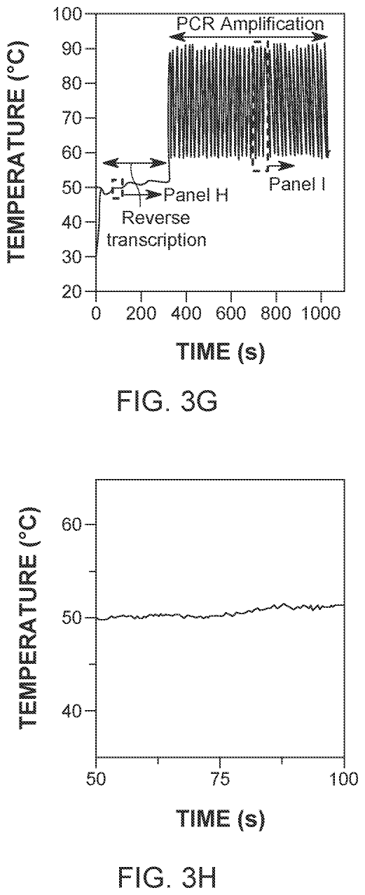

The PMNs were used as a nano-sized heater because the PMNs induced efficient PPT-based thermocycling and convenient magnetic separation. The PMN particles had a magnetic iron oxide core enclosed by a plasmonic Au shell (FIG. 3A). The iron oxide core prepared by solvothermal reaction was first functionalized with an amine-terminated surface by an organosilane. Then, small AuNPs were attached to the surface of the core through electrostatic assembly, and an Au shell was then formed.

To increase the stability of PMNs and minimize non-specific interactions during amplification, PMNs were finally modified with methoxy-polyethylene glycol (mPEG)-thiol. The elemental mapping image indicated that the core was entirely encapsulated by the shell (FIG. 3B). Also, PMNs exhibited a strong and broad plasmon wavelength, which corresponds to the peak wavelength of IR-LED (FIG. 3C). Magnetic measurements also confirmed that PMNs had excellent magnetic properties after shell coating with a magnetic moment of 4.75 emu/g (FIG. 3D). When PMNs were used in a PPT-based optical device, the temperature of the particle-containing solution increased dramatically under IR-LED lighting, while there was no noticeable change in temperature of the solution without PMNs (FIG. 3E).

By adjusting the concentration of PMNs, optimal thermocycling efficiency was achieved with 16 OD PMNs, which corresponded to the heating and cooling rates of 8.22±0.24° C./s and 5.27±0.14° C./s, respectively (FIG. 3F). In these optimal conditions, the temperature profile, which includes an isothermal process (5 minutes at 50° C.) and 40 cycles between 90° C. (0 second) and 60° C. (8 seconds), was completed within 20 minutes (FIGS. 3G, 3H, and 3I).

Through testing using DENV2 RNA, the present inventors confirmed that the effect of PMNs on amplification was negligible in the case of commercially available PCR equipment. This thermocycling protocol produced sufficient amplicons even at a low target concentration in the device of the present invention. To increase the signal-to-noise ratio for subsequent colorimetric detection, related parameters, such as the pH value, the concentrations of SGI and TMB, the LED power, and the irradiation time, were further optimized. As a result, the colorimetric detection was completed within 4 minutes, which includes a PMN collection time (less than 1 minute), a blue light irradiation time (2 minutes), and a signal measurement time (less than 1 minute). Making use of ultrafast PPT-based thermocycling and a simple colorimetric strategy, the overall assay time was less than 54 minutes (30 minutes for commercial RNA extraction, less than 20 minutes for thermocycling, and less than 4 minutes for signal detection).

In other words, when a virus was detected using the PPTRTPCR platform of the present invention, it can be seen that the virus was able to be visualized through a colorimetric reaction, and also be rapidly detected within an hour.

### 2-3. Evaluation of Analytical Sensitivity and Specificity of DENV-Specific PPT-RTcPCR

In this example, the limit of detection (LoD) for PPT-RTcPCR was investigated using serial dilutions of DENV2 IVT RNA, and compared to the reference real-time RT-qPCR. In this example, the mean OD650 of nine negative samples was 0.39±0.046, and the threshold for positivity was an OD650 of 0.53 (three-fold standard deviation of the blank sample).

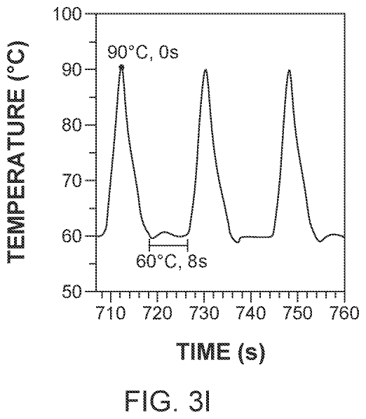

As a result of this experiment, the limit of detection for PPT-RTcPCR was estimated to be approximately 1.9 copies/μL, which was comparable to that of the reference real-time RT-qPCR (2.1 copies/μL) (FIG. 4A). The amplification signals generated by PPT-RTcPCR increased as the concentration of the target increased and reached a plateau when the concentration of RNA was higher than 10⁴ copies/μL, which may be limited by the concentrations of SGI and TMB (FIG. 4A). Also, in the samples containing 1 copy/μL of DENV IVT RNA, negative results were obtained because the amplification signals were lower than the threshold for the PPT-RTcPCR and reference real-time RT-qPCR.

As a result, an excellent linear relationship was obtained when the target concentration ranged from 1 copy/μL to 10⁴ copies/μL using the correlation equation: A=0.47±0.21 log C (where A is the absorbance at 650 nm and C is the target concentration, and R2=0.99).

Ten-fold serial dilutions of viral RNA extracted from normal human serum samples to which DENV2 was added were used to assess analytical sensitivity in a more clinically relevant environment. As shown in FIG. 4B, the PPT-RTcPCR exhibited analytical sensitivity comparable to the reference real-time RT-qPCR. For the PPT-RTcPCR, all the samples containing 11.5 FFU/mL of DENV particles were detected as positive, whereas none of the samples containing 1.15 FFU/mL of the DENV particles were detected as positive.

Therefore, excellent linearity (R2=0.99) was obtained between 1.15×10⁴ FFU/mL and 11.5 FFU/mL using the correlation equation: A=0.44±0.19 log C, indicating that the PPT-RTcPCR has similar analytical performance compared to the reference real-time RT-qPCR. The calculated LoD based on DENV2 titers was approximately 3.42 FFU/mL.

In the present invention, the analytical specificity of PPT-RTcPCR was evaluated by testing viral RNA extracted from TCF containing DENV1, DENV4, ZIKV (flavivirus), CVB3 (picornavirus), and FIPV (coronavirus). All reactions were negatives with absorbance signals below the threshold, indicating the high analytical specificity of PPT-RTcPCR (FIG. 4C).

### 2-4. Validation of Clinical Performance of DENV-Specific PPT-RTcPCR

In this example, in order to determine the diagnostic performance of DENV-specific PPT-RTcPCR, a panel of 158 human plasma samples collected from clinically suspected dengue fever patients was tested, and compared with those of the reference real-time RT-qPCR assay that was run under the same conditions.

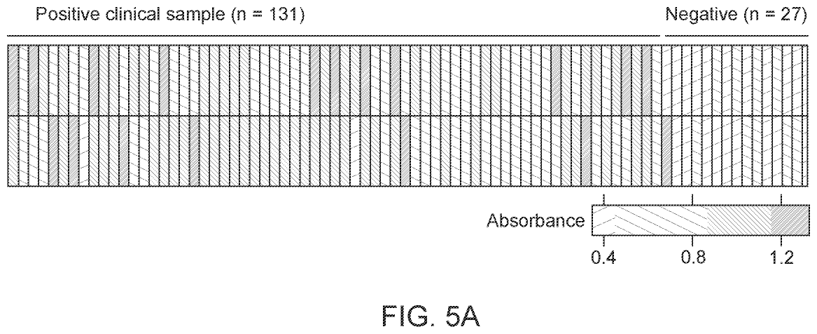

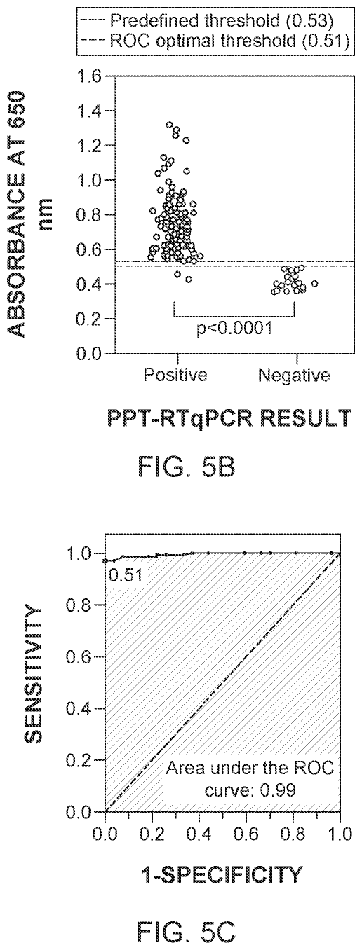

Using the predefined absorbance threshold of an OD650 of 0.53, the PPT-RTcPCR detected DENV2 positivity in 125 out of the 131 positively confirmed samples, whereas the reference RT-qPCR detected all 131 samples as positive. In the PPT-RTcPCR and reference RT-qPCR assays, all 27 negative samples were also determined as true negative (FIGS. 5A and 5B).

The clinical sensitivity and specificity of PPT-RTcPCR confirmed in this example were 95.4% (95% CI, 90.4% to 97.9%) and 100% (95% CI, 87.5% to 100%), respectively.

To rule out the possibility that the threshold calculated based on the 30 principle makes a difference in sensitivity observed between the PPT-RTcPCR and the reference RT-qPCR, the optimal threshold was recalculated using receiver operating characteristic (ROC) analysis. As a result, as shown in FIG. 5C, the optimal threshold was 0.51 with an area under the curve (AUC) of 0.99. The ROC optimal threshold was highly correlated with the set of samples. In the present invention, decreasing the threshold from 0.53 to 0.51 resulted in higher sensitivity. The analytical and clinical performance of PPT-RTcPCR was re-evaluated using the ROC optimal threshold. The calculated LoD for viral nucleic acid- and DENV2-spiked human sera were shown to be approximately 1.6 copies/μL and 2.69 FFU/mL, respectively.

TABLE 1
Comparison of diagnostic performance between
DENV2 PPT-RTcPCR and real-time RT-qPCR assays

| | | DENV2 RT-qPCR | | | Sensitivity | Specificity | Agreement |
|---|---|---|---|---|---|---|---|
| | | Positive | Negative | Total | (%, 95% CI) | (%, 95% CI) | (%, 95% CI) |
| DENV2 | Positive | 127 | 0 | 125 | 97.0 (92.4-98.8) | 100 (87.5-100) | 97.5 (83.4-99.7) |
| PPT- | Negative | 4 | 27 | 33 | | | |
| RTcPCR | Total | 131 | 27 | 158 | | | |

Given these results, the PPT-RTcPCR identified 127 of the 131 positive samples as true positive and determined all 27 negative samples as true negative, showing a clinical sensitivity and specificity of 97.0% (95% CI, 92.4% to 98.8%) and 100% (95% CI, 87.5% to 100%), respectively, compared to the reference real-time RT-qPCR (Table 1). There were four samples that tested false negative by PPT-RTcPCR, which can be potentially attributed to an extremely low concentration of the target RNA with the colorimetric signal being below the limits of detection by the spectrophotometer. The overall agreement value between the PPT-RTcPCR and the reference RT-qPCR was 97.5% (95% CI, 83.4% to 99.7%; κ=0.92), indicating that the PPT-RTcPCR had excellent diagnostic accuracy.

When the polymerase chain reaction (plasmonic photothermal reaction-based RTcPCR (PPT-RTcPCR)) of the present invention is used, a target gene can be detected quickly within 1 hour.

Also, according to the polymerase chain reaction of the present invention, the target gene can be detected with high specificity and sensitivity, thereby reducing the rate of false positives or false negatives.

Further, the present invention has an advantage in that a colorimetric reaction occurs when the target gene is present, which makes it possible to check the results with the naked eye.

So far, the present invention has been shown and described with reference to preferred embodiments thereof. Those skilled in the art to which the present invention pertains will appreciate that the present invention can be implemented in modified forms without departing from the essential features of the present invention. Therefore, the disclosed embodiments should be considered in a descriptive sense only and not for purposes of limitation. The scope of the present invention is shown in the claims rather than the foregoing detailed description, and all differences within the scope will be construed as being included in the present invention.

What is claimed is:

1. A plasmon-based reverse transcription polymerase chain reaction for detecting a target gene, comprising:
i) mixing a target gene, a primer set, and plasmonic nanoparticles and then performing a reverse transcription polymerase chain reaction; and
ii) mixing 3,3′,5,5′-tetramethylbenzidine with the product obtained in step i),
wherein SYBR Green I is added in step i) or ii).

2. The plasmon-based reverse transcription polymerase chain reaction of claim 1, further comprising, after step ii):
iii) irradiating a blue LED.

3. The plasmon-based reverse transcription polymerase chain reaction of claim 2, wherein when the product has a blue color in step iii), it is determined that the target gene is present.

4. The plasmon-based reverse transcription polymerase chain reaction of claim 1, wherein the plasmonic nanoparticles have a core-shell structure consisting of a core including iron oxide (FeO); and a shell including gold (Au) attached to a surface of the core.

5. The plasmon-based reverse transcription polymerase chain reaction of claim 4, wherein polyethylene glycol is bound to the gold particles of the plasmonic nanoparticles.

6. The plasmon-based reverse transcription polymerase chain reaction of claim 1, wherein the target gene is a viral gene.

7. The plasmon-based reverse transcription polymerase chain reaction of claim 6, wherein the viral gene is derived from at least any one virus selected from the group consisting of dengue virus, picornavirus, flavivirus, Zika virus, Powassan virus, Chikungunya virus, enterovirus, respiratory syncytial virus (RSV), Rift Valley fever, influenza virus, Tacaribe virus, Mayaro virus, West Nile virus, Yellow fever virus, and coronavirus.

8. A composition for plasmon-based reverse transcription polymerase chain reaction analysis, comprising plasmonic nanoparticles, SYBR Green I, and 3,3′,5,5′-tetramethylbenzidine.

9. The composition of claim 8, wherein the plasmonic nanoparticles have a core-shell structure consisting of a core including iron oxide (FeO); and a shell including gold (Au) attached to a surface of the core.

10. The composition of claim 9, wherein polyethylene glycol is bound to the gold particles of the plasmonic nanoparticles.

11. The composition of claim 8, wherein the SYBR Green I is added before or after performing the polymerase chain reaction, and the 3,3′,5,5′-tetramethylbenzidine is added after the polymerase chain reaction.

12. A kit for detecting a target gene, comprising the composition of claim 8.

13. The kit of claim 12, wherein the kit develops a blue color when the target gene is present.

14. The kit of claim 12, wherein the target gene is a viral gene.

15. The kit of claim 14, wherein the viral gene is derived from at least any one virus selected from the group consisting of dengue virus, picornavirus, flavivirus, Zika virus, Powassan virus, Chikungunya virus, enterovirus, respiratory syncytial virus (RSV), Rift Valley fever, influenza virus, Tacaribe virus, Mayaro virus, West Nile virus, Yellow fever virus, and coronavirus.
